(19) United States
(12) Patent Application Publication
(10) Pub. No.: US 2012/0094306 A1
(43) Pub. Date: Apr. 19, 2012

(54) PREDICTIVE EVALUATION OF THE RESPONSE TO TAXANE-INCLUDING CHEMOTHERAPY

(75) Inventors: Giovanni Scambia, Rome (IT); Maria Gabriella Ferrandina, Rome (IT); Cristiano Ferlini, Rome (IT)

(73) Assignee: UNIVERSITA CATTOLICA DEL SACRO CUORE, Milan (IT)

(21) Appl. No.: 13/378,776

(22) PCT Filed: Jun. 16, 2009
(86) PCT No.: PCT/EP2009/057461

§ 371 Date: Dec. 16, 2011

(51) Int. Cl. G01N 33/53

(52) U.S. Cl. 435/7.1

(57) ABSTRACT

The present invention relates to methods and kits for a predictive evaluation of the effectiveness of a taxane-including treatment of a tumour or of cancer cells.

The present invention was granted a foreign filing license issued by the Ministero delle Attività Produttive, D.G.S.P.C. UIBM, on the 20 of May 2009, Protocol number 46238.

The present description discloses an in vitro method for the predictive evaluation of the response to taxane-including chemotherapy of a cancer in a patient or of in tumour cells, comprising the step of determining the level of class V β-tubulin protein in a cancer sample of said patient or in said tumour cells. The description also relates to kits for an in vitro predictive evaluation of the response to taxane-including chemotherapy of a cancer in a patient from a biological sample of said cancer or in tumour cells.

### STATE OF THE ART

Cancer is one of the leading causes of deaths in most countries. The treatment of cancer often involves the systemic administration of cytotoxic compounds to the patient suffering with the disease. Unfortunately the administration of these compounds has a lot of side effects as weight loss, diarrhoea, nausea, and hair loss to more severe side effects such as anaemia, secondary cancers, organ toxicity, and even death.

The main hindrance to a successful medical treatment of cancer is drug resistance. Despite the advances obtained in the field of cancer therapy many patients, even those responding to a first line chemotherapy, fail to further respond when the disease relapses and in some cancer cases, with particular aggressive features, patients do not even respond to said first line treatment.

The failure to predict which patients will respond to a given treatment exposes all patients to severe, adverse side effects linked to chemotherapy also in the cases when, being not respondent to the chemotherapeutics, the patient will not benefit from the treatment. In order to develop new strategies able to overcome these phenomena and subsequent disease progression, many efforts have been focused on understanding the molecular mechanisms underlying drug-resistance.

It has been found out that several cancers respond differently to various chemical substances or mixtures thereof, thus rendering the predictive evaluation of the cancer treatment effectiveness on a patient a very difficult task often highly tissue specific or strictly related to the isoform or isotype of the analysed marker. Currently taxanes (such as paclitaxel, docetaxel) and vinca alkaloids (such as vinblastine, vincristine, vindesine, vinorebline, vinflunine) are among the most widely used and among the few accepted cytotoxic anticancer drugs. These drugs are potent inhibitors of cell replication whose action results in the disruption of microtubule dynamics (Schiff, P. B., and Horwitz, S. B. (1980) Proc. Natl. Acad. Sci. U.S.A. 77, 1561-1565). Microtubules are polymers of α- and β-tubulin dimers.

Among the factors of drug resistance in solid tumours, a prominent role has been assumed by the ectopic expression of β-tubulin isotypes. In fact, several tubulin isotypes are expressed in eukaryotic cells. The percentage of variability differs from species to species, but is largely conserved throughout evolution, amongst the most variable tubulin isotypes are β-tubulin III, V and VI. Tubulin isotypes, differ mainly in their C terminal region and are present in plants, insects, and in the entire chordata phylum, including mammals. In human, 18 tubulin isotypes have been reported: 9 for β-tubulin and 9 for α-tubulin. At the time of their discovery, tubulin isotypes seemed related to a given set of tissues, and the original class distinction was established following this criterion, so that the isotype most expressed in all tissues was class I, the neuronal type was class II, and the additional tissue-specific isotypes were thereafter numbered sequentially, in an attempt to correlate the gene to its expression in a specific set of tissues (in a manner similar to cytokeratins). Unfortunately, due to the overlapping spectrum of expression, this classification nowadays gives results incoherent with the original intent. An exhaustive review on tubulins and cancer drug resistance has been made by Ferlini et al. in 2007. In many tissues it is likely that the high variability in tubulin isotypes confers plasticity and extreme dynamicity to microtubules, for example in neuronal cells and in the testis. However, the expression of a given isotype is not static, since it could react to stimuli coming from environmental factors.

Some publications indicate that changes in the abundance of β-tubulin isotypes confer differential drug responses perhaps as a result of changes in drug binding affinity and/or microtubule dynamics. For example, resistant cells tend to increase class III β-tubulin (TUBB3) levels, as they do when treated with drugs that do not directly interact with microtubules (Gan et al., Cancer Res 2007), such as cisplatin and doxorubicin. Moreover, TUBB3 expression is enhanced by oxidative stress, thereby suggesting that it represents an element in a survival adaptation to hypoxia (Raspaglio et al., Gene 2008).

It also has been shown that paclitaxel (Taxol) is not effective in treating cells expressing class II β-tubulin (Haber et al. J. Biol. Chem. 270(52):31269-31275, 1995).

On the other hand, the connection between class V β-tubulin and drug resistance appears to be highly controversial.

In March 2004, Bhattacharya et al. found that a modest increase in β-tubulin V protein is associated with paclitaxel resistance on an in vitro model of cells transfected with a vector coding for mouse β-tubulin V wherein an exogenous epitope was added at the C terminal of the protein, suggesting in a cellular model, that over expression of this isotype could induce a paclitaxel-resistant phenotype. On the contrary, patent applications WO2006/076100 and WO 2006/063135 (priority date December 2004), disclosing a method of identifying a patient with cancer suitable for treatment with a specific chemical compound, disclose that amongst all the β-tubulin isotypes analyzed, the susceptibility of breast, lung and ovarian cancer to halicondrin B and hemiasterlin analogous significantly correlates with β-tubulin III levels (tab.6 of the above cited patents). In the same table, β-tubulin V levels are indicated as non significant for resistance to paclitaxel and vinblastin.

Still later, it was found (Hiser et al. 2006) that in several tumour cell lines such as colon cancer, HCT-15; breast cancer, T-47D; lung cancer, A549 and HOP-18; melanoma, Malme-3M and SK-MEL-2; ovarian, OVCAR-3 and SK-OV-3 there was no correlation between the amount of class V β-tubulin mRNA expression and the resistance to paclitaxel. whereas the same authors found a significant correlation between mRNA levels of β-tubulin III-IVa and paclitaxel resistance in lung, ovarian and prostate cancer.

From the above, it is evident that it would be useful to identify further molecular markers that could indicate whether a certain therapy will be effective or not in a given patient for a given tumour, thus avoiding to submit patients to ineffective treatments and, at the same time, pointing out those patients for which treatment could be of use.

Accurate prediction of the response of a cancer patient to chemotherapy could help to select the most efficient and appropriate drug for the cancer treatment in the patient, providing a means of individualized patient care. Given the high number of ineffective cancer treatments, there is a need in the art for reliable methods of predicting the response of cancer patients to chemotherapeutic agents. Last but not least, although a plethora of drug resistance mechanisms have been described, only few have been validated in clinical trials.

### PATENT LITERATURE CITED

WO 2006/076100
WO 2006/063135

### NON PATENT LITERATURE CITED

1. Schiff, P. B., and Horwitz, S. B. (1980) Proc. Natl. Acad. Sci. U.S.A. 77, 1561-1565
2. Ferlini et al. (2007), Current Cancer Drug Tests, 7, 352-334.
3. Gan et al, (2007), Cancer Res, 67:9356-6.
4. Raspaglio et al (2008), Gene 409:100-8.
5. Haber et al (1995), J. Biol. Chem. 270:31269-31275.
6. Bhattacharya et al (2004), Mol Biol Cell, 15: 3123-3131.
7. Hiser et al. (2006), Cell Motility and the Cytosckeleton 63: 41-52.
8. Banerjee et al. (2008), Cell Motility and Cytoskeleton 65:505-14.

### DESCRIPTION

The present invention proves for the first time, on clinical samples of patients with known medical outcome to chemotherapy, that tubulin beta class V (TUBB6) is a marker indicating a resistance of tumours and cancer cells to taxane-including chemotherapy.

Although the prior art was highly controversial on the definition of the role of β-tubulin V as a significative marker for drug resistance, and no clinical use of said controversial data was shown or suggested, the present description demonstrates for the first time that β-tubulin V is, indeed a significative marker for tumour and cancer cells resistance to taxane-including chemotherapy, and teaches for the first time how to use in practice said marker for a predictive evaluation of the effectiveness of a medical treatment.

The description hence provides an in vitro method for the predictive evaluation of the response to taxane-including chemotherapy in a mammalian patient affected by a tumour including human or in mammalian cancer cells including human comprising the steps of:

a. detecting the percentage of tumour cells expressing the protein tubulin beta of class V in a tumour sample from said patient or in a sample of said cancer cells;

b. assessing a predictive evaluation of the response to taxane-including chemotherapy in said patient or in said cancer cells, wherein a tubulin beta class V expression in percentage of tumour cells or cancer cells lower than about 40% indicates a susceptibility of the tumour or of the cancer to taxane-including chemotherapy whereas a tubulin beta class V expression in percentage of tumour cells or cancer cells higher than about 40% indicates a drug resistance of the tumour or of the cancer taxane-including chemotherapy and a kit for the predictive evaluation of the response to taxane-including chemotherapy in a mammalian patient including human or in mammalian cancer cells including human comprising tools for detecting the cells expressing the protein tubulin beta of class V in a tumour sample from said patient or in a sample of said cancer cells and instructions for assessing a predictive evaluation of the response to taxane-including chemotherapy in said patient or in said cancer cells.

In the present description the authors have demonstrated for the first time without doubt and on samples that are not in vitro transfected cells showing an artificially induced over expression of an exogenous mutated gene or cancer cells cultured in vitro, that β-tubulin V is a marker significantly related to a medical treatment, namely, that the expression of said molecule over a certain threshold allows a predictive evaluation of the effectiveness or non effectiveness of a treatment including taxane/s. In fact, using a clinical subset of samples derived from 56 ovarian cancer patients, the inventors have demonstrated, in a quantitative and reproducible way, that patients with class V β-tubulin staining in over 40% of the cells have a worst outcome with a median overall survival (OS) of 50 months with respect to the 96 months noticed in patients with no or low (below 30% of the cells) expression of class V β-tubulin. All the patients here studied belonged to a clinical cohort in which the slide was collected at the first surgery and thereafter the patients underwent a standard taxane-including chemotherapy.

The results herein provided allow for the first time to effectively use β-tubulin V (coded by TUBB6 gene) for the prediction of the effectiveness of a taxane including treatment on a tumour or on cancer cells.

### DETAILED DESCRIPTION OF THE FIGURES

FIG. 1 Representative dot-blot validation for anti-class V β-tubulin antibody. Reactivity of the tested antibody was checked in recombinant protein obtained through the cloning of the C-terminus of the selected β-tubulin isotypes with MBP (maltose-binding protein). Recombinant proteins were expressed in E. coli and then purified through column filled with Amylose resin column. In each lane at least 20 μg of the recombinant protein fused with the carrier MBP was spotted. The positive control was represented by anti-MBP. As depicted, anti-class V β-tubulin antibody reacts specifically only with this tubulin isotype.

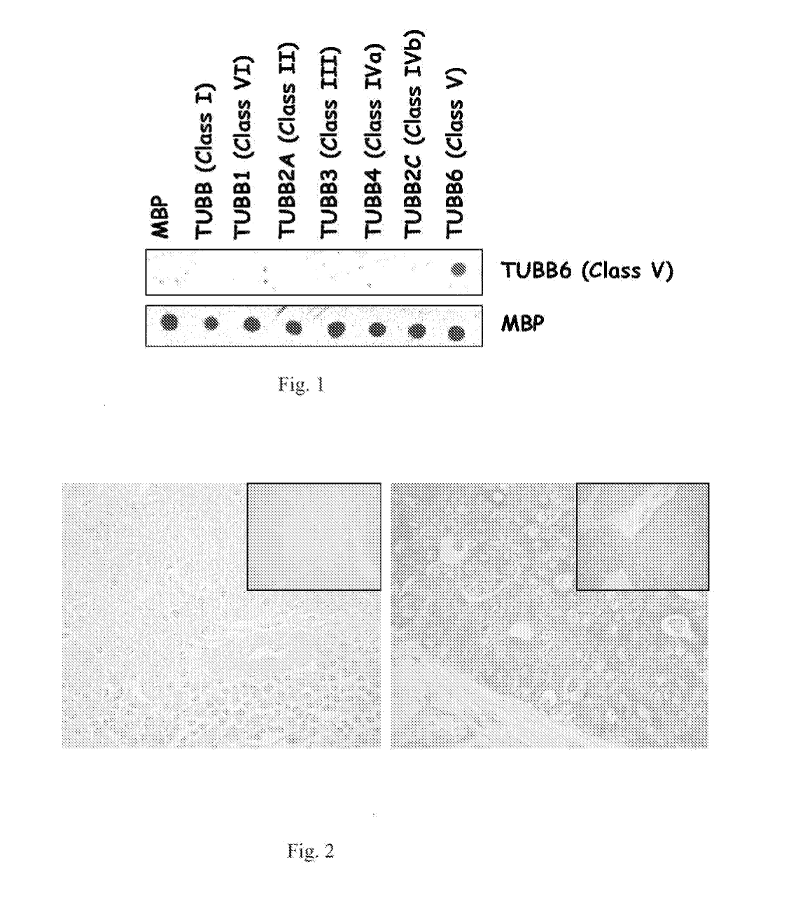

FIG. 2 Representative microscopic analysis of cases with low and high class V β-tubulin expression. Images were acquired at low (insert) and high magnification. Cell staining is cytoplasmic and in the stroma the endothelial structures are always stained also in the cases in which tumour cells are negative, thereby making the endothelial structures present in the sample, useful as internal controls for staining efficiency.

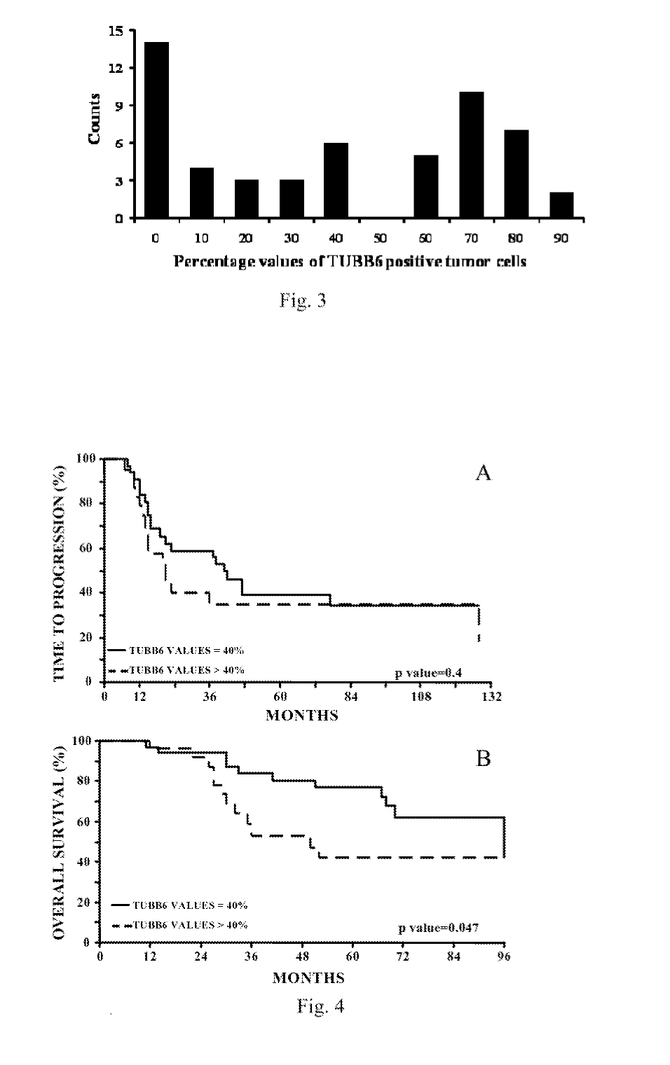

FIG. 3 Bar chart reporting the absolute count of class β-tubulin cells in the slides used in the clinical cohort used in this study. For each patient the number of positive cells was assessed and the number of patient having that level of expression is reported to have the population density for each given level of expression. The level of expression were grouped at a level of 10%, (0%=0-10%; 10%=10-20%; 30%=30-40%; 40%=40-50%; 50%=50-60%; 60%=60-70%; 70%=70-80%; 80%=80-90%; 90%=90-100%).

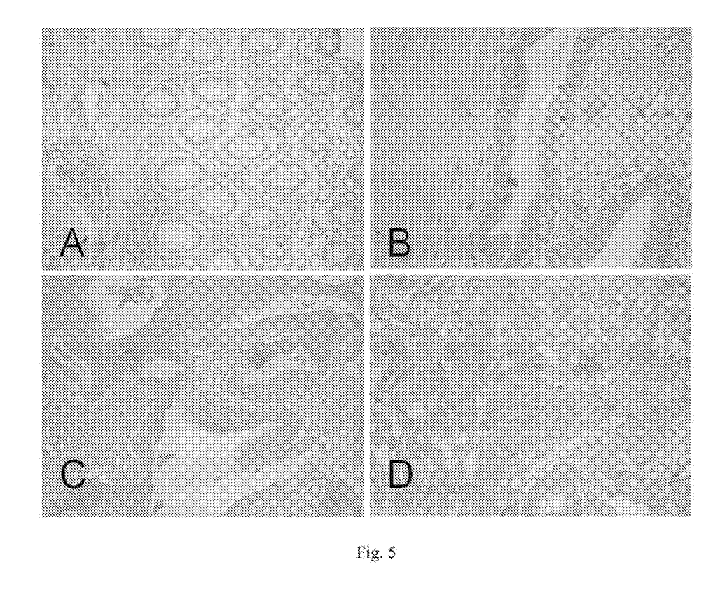

FIG. 4 Kaplan Meier curves representing TTP (A) and OS (B) according to low (continuous line) versus high (dashed line) class V β-tubulin expression content in advanced ovarian cancer patients. In A there was no statistically significant difference between the two curves. In B, the difference was statistically significant and patients having high and low levels of expression of class V β-tubulin exhibited an OS of 50 and 96 months, respectively.

FIG. 5 Representative microscopic analysis of tissue arrays immunostained with the anti-class V β-tubulin. A, B, C and D represent normal colon, normal prostate, prostate cancer and colon cancer, respectively. In A and B only stromal elements were immunostained, while in C and D a strong immunoreactivity was evident throughout the section. Also the additional specimens represented in the tissue array and corresponding to 4 different patients (normal and cancer) exhibited the same pattern of staining.

### GLOSSARY

An “in vitro method” is commonly and herein as well, a method carried out outside of a living organism as opposed to in vivo which is a method carried out inside or on a living organism.

“Predictive evaluation” means, in the present description and claims, an evaluation that can be made before carrying out a determinate action (in this case an in vivo treatment or in vitro assay as examples) and that allows predicting the physiological or biological effect to be expected by that action. Since medicine expects that the response to a chemotherapeutic agent(s) will be variable depending on the individual's background or its condition, it is clear that an estimate a priori, of the result(s) of a medical treatment or assay, might prove in some cases to be incorrect; this is why, the description of the invention refers to “predictive evaluation” emphasizing the predictive character of the evaluation itself.

By “taxane-including chemotherapy” a chemotherapy using one or more taxane or derivative thereof, and, optionally one or more further anti tumour drug is intended in the meaning of the present description and claims.

“Detecting cells expressing β-tubulin V protein” indicates that the detection according to the description and the claims has to reveal the expressed protein itself and does not include means for indirect detection of the expression thereof such as mRNA detection.

“Antibody” as herein used, includes immunoglobulin molecules and antigen binding fragments thereof. The antibody can be a polyclonal antibody or a monoclonal antibody. The antibody can be labelled by a detectable means and includes enzymatically, radioactively, fluorescently, chemiluminescently or bioluminescently labeled antibodies by any of the many different methods commonly known to those skilled in this art.

“Active fragments of an antibody” or “active fragment thereof” when referred to an antibody specifically binding tubulin beta of class V, such as the antibody herein described, indicates antigen binding fragments as specified below wherein said fragments maintain the same binding specificity of the antibody from which they derive.

By “antigen-binding fragments” it is intended to encompass fragments such as Fv, Fab, F(ab′)2, Fab′, scFv, ScFv-Fc retaining the specificity for tubulin beta of class V binding as for the active fragments above. These antibody fragments are obtained using conventional techniques well known to those with skill in the art, and the fragments are screened for utility in the same manner as are intact antibodies.

### DETAILED DESCRIPTION OF THE INVENTION

The present description hence discloses an in vitro method for the predictive evaluation of the response to taxane-including chemotherapy in a mammalian patient affected by a tumour including human or in mammalian cancer cells including human comprising the steps of:

a. detecting the percentage of tumour cells expressing the β-tubulin V protein in a tumour sample from said patient or in a sample of said cancer cells;

b. assessing a predictive evaluation of the response to taxane-including chemotherapy in said patient or in said cancer cells, wherein the β-tubulin V protein expression in percentage of tumour cells or cancer cells lower than about 40% indicates a susceptibility of the tumour or of the cancer to taxane-including chemotherapy whereas the β-tubulin V protein expression in percentage of tumour cells or cancer cells higher than about 40% indicates a drug resistance status of the tumour or of the cancer cells to taxane-including chemotherapy.

According to the method above, the sample can be a tissue sample of a biopsy, mounted on any way known to the skilled person, fresh or preserved, and the tumour cells can either be free tumour cells deriving from a patient, or cells of a tumour cell line. Specimens include but are not limited to standard paraffin embedded slides normally used in standard diagnostic pathology after a process of: preparation of sections of 3 μm thick; mounting of sections onto poly-L-lysine coated slides; drying of the slides at 37° C. overnight; rehydration in xilene and blocking of the endogenous peroxidase with 3% H₂O₂ in distilled water for 5 min; reduction of non specific binding through incubation with 20% normal rabbit serum for 25 min. at room temperature.

The detection described can be carried out with any protein detection method known in the art allowing to count the cells expressing the desired protein and the cells not expressing the said protein, or, allowing, anyhow, to establish a percentage of expression of the protein in the sample analysed. This includes but is not limited to visual inspection and microscopic analysis as well as automatic image analysis performed with dedicated software on the images obtained with microscopy.

The detection shall enable the person carrying out the method described herein, to assess, directly (i.e. by direct count) or indirectly (i.e. by calculating an average of cells per area and assessing the percentage of said area in which the protein of interest can be detected), a percentage of tumour cells expressing the protein.

Such methods include but are not limited to immunohistochemistry, wherein the count is made, by way of example, counting the stained cells (expressing the protein tubulin beta of class V) or calculating through image analysis and a dedicated software the threshold above which a given signal is positive and then the area percentage, in a given image, exhibiting a signal above the threshold.

In general, in an “immunohistochemical method” a section of tissue is tested, by way of example, by exposing the tissues to antibodies that are specific for the protein that is being assessed. The antibodies are then visualized by any of a number of methods to determine the presence and amount of the protein present. Examples of methods that may be to visualize antibodies commonly known include but are not limited to the use of, luciferase, alkaline phosphatase, horseradish peroxidase or P-galactosidase or chemical methods such as DAB/Substrate chromogen, gold, fluorescent or labelled antibodies by any of the many different methods known to those skilled in this art.

In an embodiment of the immunohistochemical method (and the kit for carrying out the said method) according to the present description and claims, detection or assaying the level of β-tubulin V protein in a tumour sample or in cancer cells includes contacting said sample or said cells with an antibody, or with an antigen-binding fragment thereof specific for class V β-tubulin protein or a fragment thereof and determining the amount of the binding antibody on said tumour, or on said cancer cells. As stated above, “antibody” includes immunoglobulin molecules and active binding fragments thereof, wherein the protein bound by the antibody and by its fragment is the same, hence the antibody and the active fragment thereof will be both specific for tubulin beta of class V. The antibody can be a polyclonal antibody or a monoclonal antibody. The antibody can be labelled by a detectable means and includes enzimatically, radioactively, fluorescently, chemiluminescently or bioluminescently labelled antibodies by any of the many different methods known to those skilled in this art.

By “active fragments thereof” fragments such as Fv, Fab, F(ab′)2, Fab′, scFv, ScFv-Fc, specifically binding tubulin beta of class V are included.

The tubulin beta of class V, due to the high variability of the molecule, will be the endogenous tubulin beta of class V, naturally occurring in the tissue or cells analysed. It has been reported in the art, and the authors themselves have experienced so, that antibodies against beta tubulin of class V are extremely difficult to develop, and are even species specific. Furthermore, the authors have found out that, even the species of the animal in which the antibody is developed, starting from the same epitope, might produce effective or non effective antibodies. By way of example, it has been reported (Banerjee et al., Cell Motility and Cytoskeleton 2008) that a monoclonal antibody, that was developed against class V β-tubulin of chicken in rodent, was found to be not reactive against the C terminus of human class V and the author have found that antibodies raised in rabbit against a given C terminal epitope of human V β-tubulin, were not able to recognise the human β-tubulin V.

On the other hand, once the species specific anti V β-tubulin is obtained, the active antibody fragments can be obtained using conventional techniques well known to those with skill in the art, and said fragments can be screened for utility in the same manner as are intact antibodies.

In an embodiment of the method and the kit of the present description and claims, the antibody can be an antibody developed against the C terminal epitope of a beta tubulin V protein. When the beta tubulin of class V is the human beta tubulin of class V, the antibody can be generated against the epitope CAFEDEEEEIDG and the antibody will have to show single specificity for human β-tubulin V protein. In particular, the antibody used in this study has been obtained using as immunizing peptide the sequence CAFEDEEEEIDG coupled to (Keyhole Limpet Hemocyanin) KLH as adjuvant carrier protein. The same approach was performed in 4 hens and 4 rabbits. Immunization was performed at day 0 and then repeated at interval of three weeks. After three immunisation cycles, the level of immunising response was assessed and compared with specimens collected at the day 0 for the presence of specific immune response against human β-tubulin V in eggs and sera for hens and rabbits, respectively. A signal was detectable only in 1 of the four hens immunised while any of the rabbits produced a detectable response. After detection of the signal IgY were purified against the immunogenic peptide using affinity columns and standard chromatography. Specfic antibodies were then collected, aliquoted and stored at −20° C. for subsequent use.

Interestingly, the method of the present invention can be carried out on post-surgery patient tumour samples and even on formalin fixed paraffin embedded tumour samples Alternatively, the samples may be fixed with Buin fixer or with glutaraldehyde by standard procedures. However, the preparation of the sample is not included in the present description as the method herein described is a laboratory method that can be performed in a different place and moment from the collection and fixing of the biopsy, and the kit is a kit ready to use on tumour samples or cancer cells samples in general and is not bound to a particular preparation method of the tumour sample or of the cancer cells sample. The description here intends to point out that even formalin fixed paraffin embedded samples are analysable with the method of the invention.

The tumour sample may be any tumour sample for which the presence of β-tubulin V protein is detectable despite the absence of said protein in the same tissue when non affected by tumour, in particular when said tumour shows, a variable expression level of said β-tubulin V protein. A suitable tumour sample may be, but is not limited to, ovarian, prostate and colon tumour.

The method through which assessment of β-tubulin V protein is performed requires visual morphology to ensure that expression of the biomarker is specific of cancer cells, since we were able to demonstrate expression of the protein in normal tissue in endothelial cells. For this reason, bulk proteomic assays such as western blotting and standard spectrometric analytical techniques are unsuitable for such purpose.

According to the description and the claims, a taxane-including chemotherapy, might be a chemotherapy using, as the sole active principle, a taxane or a derivative thereof such as paclitaxel or docetaxel, or a combination thereof. Moreover, a taxane including therapy according to the method of the description and the claims may further include platinum (cisplatin or carboplatin or other analogs, doxorubicin or other chemotherapeutics whose clinical use was approved in combination with taxanes).

Given the present costs for chemotherapies, it will be more likely that said treatments will be carried out on human patients, an embodiment of the invention, hence, is the method above carried out on human tumour samples or on human cancer cells, wherein, when one or more anti class V β-tubulin antibody is used, or one or more active fragment thereof as herein described or a combination thereof, all said antibody/antibodies and or fragment/s thereof will specifically bind human beta tubulin of class. V.

The present description and claims encompasses also a kit for the predictive evaluation of the response to taxane-including chemotherapy in a mammalian patient including human or in mammalian cancer cells including human comprising tools and reagents for detecting the cells expressing the β-tubulin V protein in a tumour sample from said patient or in a sample of said cancer cells and instructions for assessing a predictive evaluation of the response to taxane-including chemotherapy in said patient or in said cancer cells.

For tools and reagents for detecting the cells expressing the β-tubulin V protein in a tumour sample from said patient or in a sample of said cancer cells, reagents such as buffers, detection mixtures and reagents for the detection of protein expression on a histological sample as commonly used in the art are included. In an embodiment, the kit will comprise, as detection tool one or more antibody or active fragment thereof as described above, specific for β-tubulin V protein. Depending on the species for which the kit is produced, the antibody or active fragment thereof just mentioned will be specific for the β-tubulin V protein of said animal or animals.

In an embodiment, as previously explained, the antibody or active fragment thereof will be specific for human β-tubulin V protein.

The antibody or active fragment thereof, might be present in one or more aliquots in the kit, and will be either unlabelled or labelled in any suitable way commonly known in the art.

Non limiting examples of said labelling are cited above, can be by any detectable means and includes enzimatically, radioactively, fluorescently, chemiluminescently or bioluminescently labelled antibodies by any of the many different methods known to those skilled in the art.

Besides all the available commercial kits for antibody labelling and all the available protocols for the same, antibody labelling is also an available commercial service hence there is no need of a particularly detailed description of how to carry out an antibody labelling. Suitable kits, but not limited thereto, are DyLight™ Antibody Labeling Kits from Celbio, Amersham CyDye™ Antibody Labeling Kits, Lightning-Link™-Kits from Innova, Active Motifs Chromeo™ Antibody Labeling Kits, CHROMIS 550 Antibody. Labeling Kit, APEX Antibody Labeling Kits, LYNX Rapid Conjugation Kits, Biotin Single-Shot Antibody Labeling Kit, The MFP488 Antibody Labeling Kit and the like. The detection of the labelled antibody will be strictly dependent on the labelling system selected. The kit of the present description and claims may provide one or more vials of unlabelled and/or vials of labelled antibody anti-β-tubulin V protein, species specific according to the species for which the kit is made, and/or one or vials of unlabelled and/or vials of labelled active antibody fragment beta tubulin of class V specific, species specific according to the species for which the kit is made. Moreover, the kit may provide also aliquots of detection buffers, detection reagents, instructions for the predictive evaluation according to the data obtained. The reagents aliquots comprised in the kit may include specific reagents for treatment of tumour samples to be analysed. In particular, the kit may provide a detailed protocol of antigen retrieval procedure for paraffin embedded tissues, since clinical use is routinely associated to maintenance of specimens through the procedure of paraffin-inclusion. When the kit comprises antibody/ies and/or active fragments thereof, said antibodies or fragments can be generated against the C-terminal region of the tubulin beta of class V proteins, in an embodiment, in which one or more of the antibodies and/or fragments are against human beta tubulin of class V, said antibody or active fragment thereof are generated against the epitope CAFEDEEEEIDG.

Furthermore, in another embodiment, antibodies or active fragments thereof against human beta tubulin of class V as described above are generated in small animals such as rabbit, chicken and mouse.

In a further embodiment, the antibodies or active fragments thereof are generated as in the example below through immunisation with the peptide CAFEDEEEEIDG conjugated with KLH, checking of the specific immune response against β-tubulin V protein and purification through column affinity chromatography against the immunising peptide. Fragments can be generated using endopeptidases such as papain or others before column affinity chromatography against the immunising peptide.

The antibody fragments can be any of the kind of fragments commonly used in immunohistochemistry such as Fab fragments of the antibody obtained though papain digestion, but not limited to, the fragments listed above.

The kit according to the present description and claims, may further comprise positive and/or negative control samples as well as calibrated samples (e.g. samples wherein the protein beta tubulin of class V is expressed in a known percentage, such as about 0, 10, 20, 30, 40, 50, 60, 70, 80, 90, 100%. The word about here is used due to the possible biological origin of the sample, which can be a tumour sample, a cancer cell sample, a healthy tissue sample or a combination thereof.

The control may comprise healthy and cancer cells in a known percentage and hence provide a very reliable parameter for the measurement of the signal.

The presence of positives and/or negative controls will allow the user to verify the effectiveness of the reagents and the presence of a possible background.

The following examples summarise the clinical experiments carried out by the authors, and indicate possible embodiments of the method and kit of the description and claims and are herein enclosed for teaching a mode for carrying out the method herein described and not to limit it. The skilled person will readily understand that the following examples include standard methods unless differently specified.

### EXAMPLES

### 1. Patients and Study Design

The study included 56 ovarian cancer patients that gave their informed consent for experimental use of the clinical data and patients whose clinico-pathological characteristics are summarized in Table 1. Sixteen (28.6%) patients were >65 years old (median age=59 years, range: 35-83). Thirty-seven cases (66.1%) were stage III, and 11 (19.6%) cases were stage 1V disease. Serous histotype was documented in the vast majority (n=39, 69.6%) of cases. Maximal surgical effort has been attempted in all patients resulting in optimal debulking (apparently absent residual tumor), which underwent surgical removal of tumour masses, along with total abdominal hysterectomy, adnexectomy, radical omentectomy, appendectomy, multiple biopsies, and additional surgery (intestinal resections, diaphragm stripping) when required. In patients judged to be unresectable at first surgery because of extensive peritoneal bulky carcinomatosis, agglutinated bowel/mesentery and infiltration of the upper gastrointestinal tract and/or the major vessels, were submitted only to multiple biopsies. All patients received platinum-based chemotherapy (75-100 mg/m2 for cisplatin, AUC=5 for carboplatin, per cycle), plus paclitaxel (135-175 mg/m2 for each cycle). Response to chemotherapy was assessed according to WHO criteria.

Immunostaining for Class V β-Tubulin

Pretreatment tumor tissues biopsies were obtained at first surgery in all cases. Tissue specimens were fixed in 10% formalin and paraffin embedded according to standard procedures. Immunostaining was done on 3-μm tissue sections mounted on poly-L-lysine-coated slides and dried at 37° C. overnight. After the slides were deparaffinized in xylene and rehydrated conventionally, the endogenous peroxidase activity was blocked with 3% H₂O₂ in TBS for 5 minutes. Sections were incubated with 20% normal rabbit serum 20% for 30 minutes at room temperature to reduce nonspecific binding, then with the polyclonal chicken anti human class V β-tubulin antibody in 1% bovine serum albumin-PBS. Class V β-tubulin detection was evaluated by a labeled polymer. The EnVision-rabbit+System-HRP System (DAKO, Carpinteria, Calif.) was used. Diaminobenzidine was used as a chromogen (DAB Substrate System, DAKO). Negative controls were done by omitting the primary antibody. Results were expressed as the proportion of immunostained tumor cells.

### 2. Microscopic Analysis

The analysis of all tissue sections was done without any prior knowledge of the clinical variables by two observers in blind by means of light microscopy. The proportion of immunostained tumor cells was scored at low magnification (5× objective lens) by evaluating the entire tumor area. In case of disagreement, sections were submitted to a novel evaluation. Patients expressing immunoreactivity at a level of 0% and 100% were categorized as negative and positive, respectively. For patients exhibiting a focal immunoreactivity, the number of positive cells was carefully assessed in each section through the count of at least 5 fields at a higher magnification. The number of positive cells was then averaged. Using the data from the whole series, the median was calculated and negative patients were considered those exhibiting a percentage of immunoreactivity equal or below the median value. On the other hand, positive patients were defined as those having a percentage of immunoreactivity higher than the median value. In our clinical setting the median value was 40%.

### 3. Statistical Analysis

Fisher's exact test (or [symbol] 2 test for proportion) were used to analyze the distribution of class V β tubulin positivity according to clinico-pathological features and response to treatment. Time to progression (TTP) and OS were calculated from the date of diagnosis to the date of progression/death or date last seen. Medians and life tables were computed using the product-limit estimate by the Kaplan and Meier method and the log-rank test was employed to assess the statistical significance. Statistical analysis was carried out using SOLO (BMDP Statistical Software, Los Angeles, Calif.).

### 4. Antibody Generation

The antibody anti human β-tubulin V protein was developed in hen. Briefly, the immunogenic peptide CAFEDEEEEIDG conjugated with KLH was subcutaneously injected to four hens. Immunisation was repeated three times at interval of three weeks and one week after the last immunisation eggs were collected and IgY were checked for immune response against the panel of human β-tubulins as depicted in FIG. 1. Of the four hens, only one exhibited a significant immune response. Eggs were then collected and IgY against the antigen were purified through affinity column chromatography against the immunogenic peptide. Antibodies were then eluted with 0.2 M glycine pH=3.0 and immediately buffered in tube with fresh PBS. After overnight dialysis with fresh PBS, antibodies were aliquoted and stored at −20° C.

### 5. Class V β-Tubulin Immunostaining and Clinical Outcome

FIG. 2 shows representative examples of high class V β-tubulin immunoreaction in primary ovarian cancer. The distribution of the percentages of class V β-tubulin stained cells is shown in FIG. 3: the percentage values of class V β-tubulin positive tumour cells ranged between 0-100%, (median=40%); overall, class V β-tubulin immunoreaction was observed in 42/56 cases (75%), and the percentage of positively stained cells showed a wide range of variability (range: 5-100). In Table 1, the percentage of cases with high class V β-tubulin expression is summarized according to clinico-pathological features. Class V β-tubulin positivity was found not to be associated with any of the clinico-pathological parameters examined. No association with pathological response to chemotherapy was found. Similar results were found when analyzing the percentage of class V β-tubulin immunostained cells as a continuous variable (data not shown). Follow up data were available for all patients. After a median follow up of 53.5 months (range: 11-100) progression and death of disease were observed in 36 (64.3%) and in 23 (41.1%) cases, respectively. FIG. 3 shows the TTP and OS curves in the whole series. There was no statistically significant difference in terms of TTP in cases with high versus low class V β-tubulin expression (p value=0.4). On the other hand, cases with high class V β-tubulin expression showed a worse OS with respect to cases with low class V β-tubulin expression (median OS=50 months versus median OS=96 months) (p value=0.047). In summarizing these results, patients expressing high levels of class V β-tubulin are featured by biological aggressiveness and poor response in terms of OS to standard chemotherapy as compared to patients with low levels of class V β-tubulin.

### 6. Expression of Class V β-Tubulin Immunostaining in Additional Tissues

To verify the potential value of this antigen as biomarker in other tissues, the expression of class V β-tubulin was assessed in normal and cancer tissues using the technique of tissue array. Briefly, a commercial tissue array (normal and cancer tissues, Superbiochips, Seoul, Korea) was purchased. In the tissue array paraffin serial embedded sections of 2 mm were spotted onto slides. The advantage of the tissue array is that several specimens can be prepared altogether using the same immunoreaction. Samples were prepared and evaluated as described above. Presence of cancer cells in the tissue arrays containing cancer tissues was verified through microscopic analysis and confirmed by a certified pathologist for each given tissue. Results of interest are summarized in FIG. 5. In at least other two tissues there was a striking difference between the staining pattern of normal (FIGS. 5A and 5C) and cancer cells (FIGS. 5B and 5D). In colon and prostate normal tissue (FIGS. 5A and 5B, respectively), immunostaining was confined to stromal elements, while in the correspondent cancer specimens a strong immunoreactivity was noticed (FIGS. 5C and 5D) throughout the section. This finding suggests that class V β-tubulin expression could behave as a biomarker also in additional tissues, since expression is highly increased in cancer tissues as compared to the expression levels measured in normal tissue.

1. An in vitro method for the predictive evaluation of the response to taxane-including chemotherapy in a mammalian patient affected by a tumour including human or in mammalian cancer cells including human comprising the steps of
a. detecting the percentage of tumour cells expressing the protein tubulin beta of class V in a tumour sample from said patient or in a sample of said cancer cells;
b. assessing a predictive evaluation of the response to taxane-including chemotherapy in said patient or in said cancer cells, wherein a tubulin beta class V expression in percentage of tumour cells or cancer cells lower than about 40% indicates a susceptibility of the tumour or of the cancer to taxane-including chemotherapy whereas a tubulin beta class V expression in percentage of tumour cells or cancer cells higher than about 40% indicates a drug resistance of the tumour or of the cancer taxane-including chemotherapy.

2. The method according to claim 1 wherein the tumour is an ovarian tumour or a prostate tumour or a colon tumour.

3. The method according to claim 1, wherein said taxane-including chemotherapy includes paclitaxel and/or docetaxel.

4. The method according to claim 3 wherein said taxane-including chemotherapy further includes cysplatinum.

5. The method of claim 1 wherein said tumour sample is a paraffin-embedded sample.

6. The method of claim 1 wherein said patient or said cancer cells are human.

7. The method of claim 1 wherein the detection step a. is carried out by performing an immunohistochemical detection with ah antibody or active fragments thereof specifically binding tubulin beta of class V.

8. A kit for the predictive evaluation of the response to taxane-including chemotherapy in a mammalian patient affected by a tumour including human or in mammalian cancer cells including human, comprising tools and reagents for detecting the cells expressing the protein tubulin beta of class V in a tumour sample from said patient or in a sample of said cancer cells and instructions for assessing a predictive evaluation of the response to taxane-including chemotherapy in said patient or in said cancer cells.

9. The kit of claim 8 wherein said tools for detecting the cells expressing the tubulin beta of class V comprise an antibody or one or more active fragments thereof specifically binding tubulin beta of class V.

10. The kit according to claim 9 wherein said antibody or said active fragment thereof is generated against the epitope CAFEDEEEEIDG (SEQ ID NO: 1).

11. The kit according to claim 9 wherein said antibody or said active fragment thereof is labelled.

12. The kit according to claim 9 further comprising means for the detection of said antibody or said active fragment thereof.

13. The kit according to claim 8 further comprising positive and, optionally, negative samples on which tubulin beta of class V can be detected or non detected, respectively, with said tools.


SEQUENCE LISTING (1 sequence)

SEQ ID NO: 1 - protein, 12 aa, Homo sapiens
CAFEDEEEEIDG